(19) United States
(12) Patent Application Publication
(10) Pub. No.: US 2026/0263648 A1
(43) Pub. Date: Sep. 10, 2026

(54) Smart Device with Integrated UV-Light

(71) Applicant: Cole Reynoso, Spokane, WA (US)

(72) Inventor: Cole Reynoso, Spokane, WA (US)

(21) Appl. No.: 19/096,170

(22) Filed: Mar. 31, 2025

Related U.S. Application Data
(60) Provisional application No. 63/766,418, filed on Mar. 4, 2025.

(51) Int. Cl. A61L 2/10; G16H 40/63

(52) U.S. Cl. CPC A61L 2/10; G16H 40/63; A61L 2202/11; A61L 2202/14; A61L 2202/16

(57) ABSTRACT

A smart device with integrated UV-light is provided. The device is comprised of a smart device with an integrated UV-light module designed to enhance hygiene awareness by enabling users to detect bacteria, dirt, and stains on surfaces that appear clean under visible light. The device comprises a smart device such as a smartphone or tablet with at least one UV-light module integrated into the camera or flashlight. The UV-light module may emit UVA, UVB, or UVC wavelengths to facilitate fluorescence-based surface inspections, organic material detection, or germicidal applications. Users can activate the module through the device's flashlight settings, a physical button, or a software interface that allows adjustments in UV intensity and wavelength selection. Some embodiments include an auto-detection mode that activates the UV-light based on ambient lighting conditions or surface proximity.

### CROSS-REFERENCE TO RELATED APPLICATION

The present application claims priority to, and the benefit of, U.S. Provisional Application No. 63/766,418, which was filed on Mar. 4, 2025, and is incorporated herein by reference in its entirety.

### FIELD OF THE INVENTION

The present invention relates generally to the field of UV-light devices. More specifically, the present invention relates to a smart device, such as a smartphone or tablet, with an integrated UV-light module that emits UVA, UVB, or UVC wavelengths for surface inspections, organic material detection, or germicidal applications. Accordingly, the present disclosure makes specific reference thereto. Nonetheless, it is to be appreciated that aspects of the present invention are also equally applicable to other like applications, devices, and methods of manufacture.

### BACKGROUND

Germs, viruses, and bacteria are present on many surfaces but are not visible to the naked eye. This invisibility makes it difficult for individuals to assess cleanliness accurately, leading to unintended contact with contaminated surfaces. While UV black lights can highlight unclean areas by causing certain residues and biological contaminants to fluoresce, these lights are not commonly available for everyday use. People may unknowingly touch surfaces harboring harmful microorganisms, increasing the risk of illness and disease transmission. In public spaces, workplaces, and even at home, individuals lack an efficient method to detect contamination before interacting with surfaces. Traditional cleaning methods often rely on assumptions rather than visual confirmation, leading to either insufficient cleaning or unnecessary disinfection of already clean areas

Therefore, there exists a long-felt need in the art for a smart device with an integrated UV-light that allows users to detect bacteria, stains, and contaminants on surfaces that appear clean under visible light. There also exists a long-felt need in the art for a smart device with an integrated UV-light that enables users to selectively activate the UV function through hardware or software controls for real-time surface inspection. Moreover, there exists a long-felt need in the art for a smart device with an integrated UV-light that includes configurable UV intensity levels and wavelength selection, allowing users to tailor the device's functionality for different inspection and disinfection applications.

The subject matter disclosed and claimed herein, in one embodiment thereof, comprises a smart device with integrated UV-light. The device is comprised of a smart device with an integrated UV-light module designed to enhance hygiene awareness by enabling users to detect bacteria, dirt, and stains on surfaces that appear clean under visible light. The device comprises a smart device such as a smartphone or tablet with at least one UV-light module integrated into the camera or flashlight. The UV-light module may emit UVA, UVB, or UVC wavelengths to facilitate fluorescence-based surface inspections, organic material detection, or germicidal applications. Users can activate the module through the device's flashlight settings, a physical button, or a software interface that allows adjustments in UV intensity and wavelength selection. Some embodiments include an auto-detection mode that activates the UV-light based on ambient lighting conditions or surface proximity. Additionally, an alternative design incorporates a ring of UV modules around the camera to enable both hygiene inspections and fluorescence-based photography. The device may also feature a germicidal UVC mode, emitting short-wavelength ultraviolet light to deactivate bacteria and viruses by disrupting their genetic material.

In this manner, the smart device with integrated UV-light of the present invention accomplishes all the foregoing objectives and incorporates UV-light functionality into smart devices such as smartphones and tablets. Further, the UV-light module can be activated to provide users with an immediate and practical method for inspecting surfaces for germs and contaminants. The device also allows users to toggle between standard illumination and UV modes, enabling fluorescence-based surface inspection for identifying stains and residues. Additionally, the device may have a UVC light for germicidal applications, offering a localized disinfection feature to reduce bacterial and viral contamination. The integration of adjustable UV intensity levels and wavelength selection enhances user control, making the device adaptable for various hygiene-related applications. By incorporating UV technology into a portable and multifunctional smart device, the device provides an accessible and efficient solution for improving cleanliness and health awareness in personal, professional, and public settings.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a smart device with integrated UV-light. The smart device with integrated UV-light enhances hygiene awareness by allowing users to detect bacteria, dirt, and stains on surfaces that appear clean under visible light. This eliminates the need for a separate UV flashlight, enabling convenient surface inspections in personal, public, and workplace environments.

The device is comprised of a smart device such as a smartphone, tablet, or other portable electronic device. The device further includes at least one UV-light module, which may be integrated into the camera module, flashlight, or other hardware areas. The module can be activated through the flashlight settings or may be embedded within the flashlight itself, allowing users to switch between standard and UV illumination modes via the software interface.

The module may include LEDs emitting UVA, UVB, or UVC wavelengths, enabling fluorescence-based inspections, organic material detection, or germicidal applications. The module may contain an array of LEDs emitting specific wavelengths, such as UVA (315-400 nm) for stain detection, UVB (280-315 nm) for organic substances, and UVC (200-280 nm) for disinfection. The device may include a germicidal UVC mode for breaking down bacterial and viral genetic material. An alternative configuration features a ring of UV-light modules embedded around the camera, allowing for both surface inspection and fluorescence-based photography. The module may utilize high-efficiency UV LEDs to provide consistent illumination with minimal power consumption.

In one embodiment, the module can be activated via a physical button for quick access. The software interface allows users to adjust UV intensity levels and wavelength selection. An auto-detection mode may activate the UV-light module based on ambient lighting or surface proximity.

By integrating this functionality into consumer electronics, the device improves hygiene awareness and enables users to detect and mitigate contamination risks. This provides a practical, portable, and efficient solution for enhancing cleanliness and health standards in various settings.

The invention also includes a method of using the device. The user activates the UV-light module via the flashlight settings, a physical button, or the software interface. The module emits ultraviolet light in a selected wavelength, which can be adjusted through the software interface based on environmental conditions. The user directs the device toward a target surface for inspection and may capture images or videos for analysis, storage, or sharing. In germicidal UVC mode, the module emits short-wavelength ultraviolet light for surface disinfection. This method enables convenient contamination detection and localized disinfection, improving hygiene awareness in personal and professional environments.

Accordingly, the smart device with integrated UV-light of the present invention is particularly advantageous as it incorporates UV-light functionality into smart devices such as smartphones and tablets. Further, the UV-light module that can be activated to provide users with an immediate and practical method for inspecting surfaces for germs and contaminants. The device also allows users to toggle between standard illumination and UV modes, enabling fluorescence-based surface inspection for identifying stains and residues. Additionally, the device may have a UVC light for germicidal applications, offering a localized disinfection feature to reduce bacterial and viral contamination. The integration of adjustable UV intensity levels and wavelength selection enhances user control, making the device adaptable for various hygiene-related applications. By incorporating UV technology into a portable and multifunctional smart device, the device provides an accessible and efficient solution for improving cleanliness and health awareness in personal, professional, and public settings. In this manner, the smart device with integrated UV-light overcomes the limitations of existing UV-light devices and methods of use known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front view of a smart device of one potential embodiment of a smart device with integrated UV-light of the present invention in accordance with the disclosed architecture;

FIG. 2 illustrates a rear view of a smart device of one potential embodiment of a smart device with integrated UV-light of the present invention in accordance with the disclosed architecture;

FIG. 3 illustrates a block diagram of a software of one potential embodiment of a smart device with integrated UV-light of the present invention in accordance with the disclosed architecture; and

FIG. 4 illustrates a flowchart of a method of using one potential embodiment of a smart device with integrated UV-light of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a smart device with an integrated UV-light that allows users to detect bacteria, stains, and contaminants on surfaces that appear clean under visible light. There also exists a long-felt need in the art for a smart device with an integrated UV-light that enables users to selectively activate the UV function through hardware or software controls for real-time surface inspection. Moreover, there exists a long-felt need in the art for a smart device with an integrated UV-light that includes configurable UV intensity levels and wavelength selection, allowing users to tailor the device's functionality for different inspection and disinfection applications.

The present invention, in one exemplary embodiment, is comprised of a smart device with integrated UV-light. The smart device with an integrated UV-light improves hygiene awareness by enabling users to detect bacteria, dirt, and stains on surfaces that appear clean under visible light. This eliminates the need for a separate UV flashlight, allowing for convenient surface inspections in personal, public, and workplace environments.

The device is comprised of a smart device such as a smartphone, tablet, or other portable electronic device. Additionally, the device includes at least one UV-light module, which may be incorporated into the camera module, flashlight, or other hardware components. The module can be activated through the flashlight settings or may be embedded within the flashlight itself, allowing users to switch between standard and UV illumination modes via the software interface.

The module may include LEDs that emit UVA, UVB, or UVC wavelengths, facilitating fluorescence-based inspections, organic material detection, or germicidal applications. The module may consist of an array of LEDs designed to emit specific wavelengths, such as UVA (315-400 nm) for stain detection, UVB (280-315 nm) for identifying organic substances, and UVC (200-280 nm) for disinfection. A germicidal UVC mode may also be included to break down bacterial and viral genetic material. An alternative design features a ring of UV-light modules embedded around the camera, allowing for both surface inspection and fluorescence-based photography. The module may use high-efficiency UV LEDs to ensure consistent illumination while minimizing power consumption.

In one embodiment, the module can be activated via a physical button for quick access. The software interface allows users to adjust UV intensity levels and select specific wavelengths. An auto-detection mode may automatically activate the UV-light module based on ambient lighting or surface proximity.

By integrating this functionality into consumer electronics, the device enhances hygiene awareness and enables users to identify and address contamination risks. This provides a practical, portable, and effective solution for improving cleanliness and health standards across various environments.

The invention further includes a method of use. The user activates the UV-light module through the flashlight settings, a physical button, or the software interface. The module emits ultraviolet light at a selected wavelength, which can be adjusted via the software interface according to environmental conditions. The user directs the device toward a target surface for inspection and may capture images or videos for analysis, storage, or sharing. In germicidal UVC mode, the module emits short-wavelength ultraviolet light for surface disinfection. This method facilitates convenient contamination detection and localized disinfection, improving hygiene awareness in both personal and professional settings.

As a result, the smart device with integrated UV-light offers significant advantages by incorporating UV-light functionality into portable devices such as smartphones and tablets. The UV-light module provides users with an immediate and practical method for inspecting surfaces for germs and contaminants. The ability to toggle between standard illumination and UV modes enables fluorescence-based surface inspection for identifying stains and residues. Additionally, the inclusion of UVC light for germicidal applications supports localized disinfection, reducing bacterial and viral contamination. The integration of adjustable UV intensity levels and wavelength selection enhances user control, making the device adaptable for various hygiene-related applications. By embedding UV technology into a multifunctional smart device, the invention delivers an accessible and efficient solution for improving cleanliness and health awareness in personal, professional, and public settings. In this manner, the smart device with integrated UV-light overcomes the limitations of existing UV-light devices and methods.

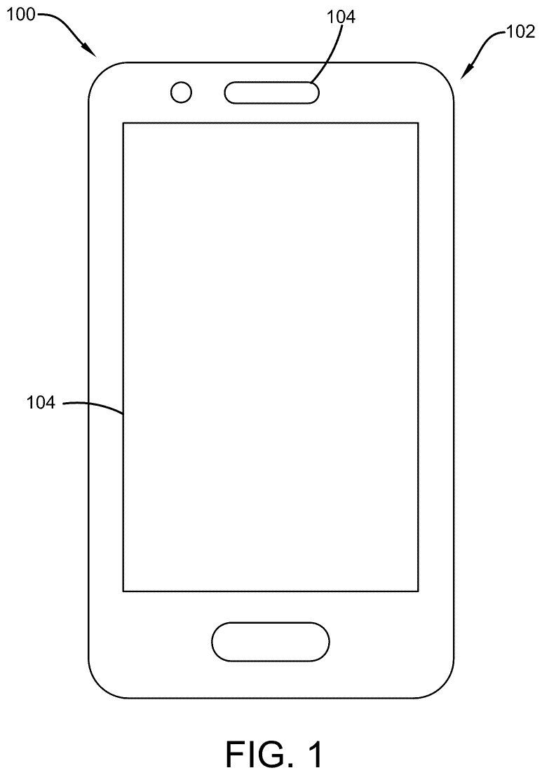

Referring initially to the drawings, FIG. 1 illustrates a front view of a smart device 102 of one potential embodiment of a smart device with integrated UV-light 100 of the present invention in accordance with the disclosed architecture. The smart device with integrated UV-light 100 is designed to enhance hygiene awareness and provide users with a convenient method for detecting bacteria, dirt, and stains on surfaces that appear clean under visible light using a smart device with integrated UV-lighting. As a result, users can instantly inspect surfaces without the need for a separate UV flashlight. The system helps improve hygiene in personal spaces, public areas, and workplaces, allowing individuals to identify contamination that may contribute to illnesses.

The device 100 is comprised of a smart device 102 such as, but not limited to, a smartphone, tablet, or other portable electronic device. The smart device 102 may have any feature/function 104 commonly found on a smart device (as seen in FIG. 1), such as, but not limited to, a touchscreen display, a processor, a memory unit, a rechargeable battery, a communication module, and an operating system capable of supporting various applications. Additional components may include a camera module, a flashlight module, biometric sensors such as fingerprint scanners and facial recognition systems, wireless communication interfaces including Wi-Fi, Bluetooth, and cellular connectivity, as well as proximity sensors, gyroscopes, accelerometers for motion detection and gesture-based controls, infrared sensors for facial recognition and night vision applications, haptic feedback mechanisms to enhance user interaction, stereo speakers for high-quality audio output, microphones with noise cancellation for improved voice recognition and communication, augmented reality (AR) and virtual reality (VR) hardware/capabilities, external device connectivity via USB, NFC, or wireless docking stations, environmental sensors that detect temperature, humidity, air quality, and ambient light levels, etc.

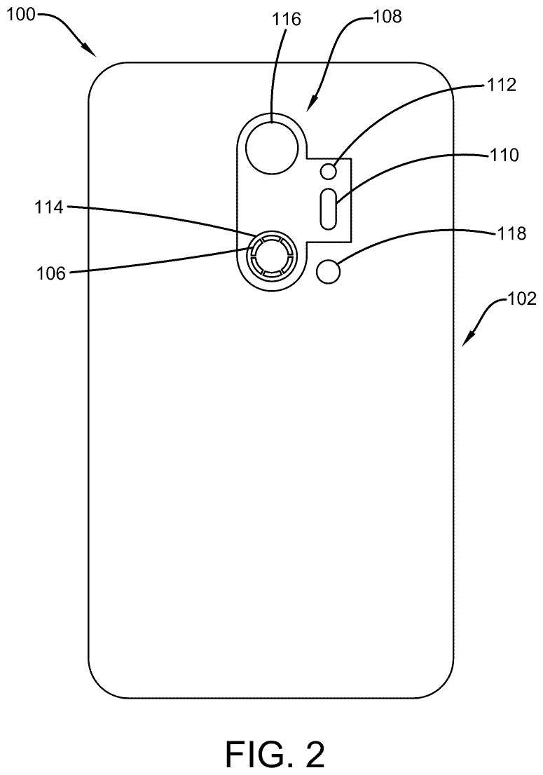

The device 100 is further comprised of at least one UV-light module 106, as seen in FIG. 2. The module 106 may be integrated into the camera module 108, flashlight 110, or other hardware component and/or area of the smart device 102. In one embodiment, the module 106 can be activated through the smart device's 102 flashlight settings. In a different embodiment, the module 106 is integrated into the flashlight 110 itself, wherein the flashlight 110 includes both a standard white LED 112 and the module 106, allowing users to toggle between standard illumination and UV inspection modes through the smart device's 102 software interface. In one embodiment, the module 106 may also include an optical filter 114 to enhance UV output while minimizing visible light interference. The UV-light module 106 may contain different LED configurations to emit UVA, UVB, or UVC wavelengths depending on the application. By way of example, UVA light is suitable for fluorescence-based surface inspections, helping users detect stains, residues, and biological contaminants that react under black light illumination. UVB light can be used for detecting certain organic substances and has limited germicidal effects. UVC light is particularly effective for germicidal applications, as it can disrupt the DNA and RNA of bacteria, viruses, and other pathogens, preventing their replication and rendering them inactive. The module 106 may be designed with an array of LEDs, wherein different sections of the module 106 emit specific wavelengths based on the selected mode, such as UVA LEDs operating between 315 and 400 nanometers for fluorescence-based inspection, UVB LEDs operating between 280 and 315 nanometers for organic material detection, and UVC LEDs operating between 200 and 280 nanometers for germicidal applications. In addition to surface inspection, the device 100 may include a germicidal UVC mode by emitting short-wavelength ultraviolet light between 200 and 280 nanometers to break down the molecular bonds in bacterial and viral genetic material. Various UV wavelengths may also be used/incorporated to identify drugs and paraphernalia as well as inspecting the magnetic security strip on paper currency for authenticity.

An alternative design features a ring of modules 106 embedded around the camera 116 of the smart device 102, enabling both surface inspection and fluorescence-based photography. This configuration appeals to users who prioritize hygiene awareness as well as photographers interested in capturing fluorescent effects. The module 106 may be constructed using high-efficiency UV LEDs that provide consistent illumination while minimizing power consumption.

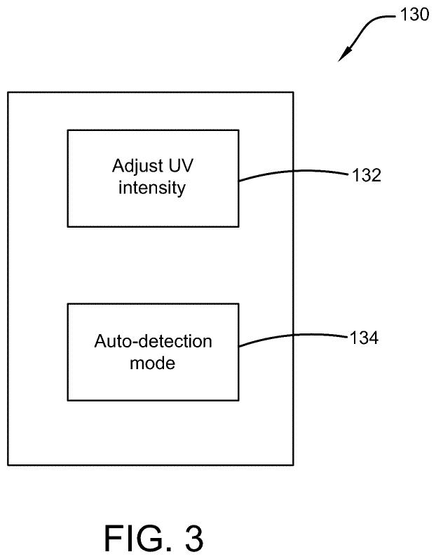

In one embodiment, the module 106 can be turned on or off via a physical button 118 on the device 102 for quick access, as seen in FIG. 2. Once activated, the module 106 illuminates surfaces, revealing stains, bacterial contamination, and other residues that are not visible under standard lighting conditions. As seen in FIG. 3, the software interface 130 of the smart device 102 may include multiple configurable UV intensity levels 132, allowing users to adjust brightness and wavelength selection based on their intended use. The system may also incorporate an auto-detection mode 134 that activates the UV-light module 106 when the device 100 detects specific conditions, such as low ambient lighting or proximity to a surface.

As a result, the device 100 represents a significant advancement in hygiene awareness and surface inspection. By incorporating this functionality into widely used consumer electronics, users can conveniently detect and potentially mitigate contamination risks in their daily environments. Therefore, the device 100 offers a practical, portable, and efficient solution for individuals and professionals seeking to improve cleanliness and health standards in various settings.

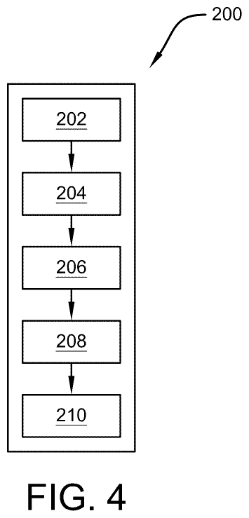

The present invention is also comprised of a method of using 200 the device 100, as seen in FIG. 4. First, a device 100 is provided, comprising a smart device 102, at least one UV-light module 106, a camera module 108, a flashlight 110, and a software interface 130 [Step 202]. Next, the user activates the UV-light module 106 [Step 204]. This can be done through the smart device's 102 flashlight settings, a physical button 118 on the device 102, or the software interface 130. Upon activation, the UV-light module 106 emits ultraviolet light of a selected wavelength (UVA, UVB, or UVC), depending on the user's needs. A user may use the software interface 130 to adjust the UV intensity 132, wavelength selection, and an auto-detection mode 134 based on environmental conditions such as low ambient lighting or proximity to a surface [Step 206]. The user then directs the device 100 toward a target surface, utilizing the emitted UV-light to inspect for bacteria, stains, dirt, and other contaminants that are otherwise invisible under standard lighting conditions. If desired, the camera module 108 can capture images or videos of the inspected surface, which can be analyzed, stored, or shared via the smart device's 102 software interface 130 [Step 208]. In an alternative mode, the device 100 may be switched to germicidal UVC mode, wherein the module 106 emits short-wavelength ultraviolet light between 200 and 280 nanometers to break down the molecular bonds in bacterial and viral genetic material, helping to disinfect the surface [Step 210]. As a result, the device 100 allows users to conveniently inspect surfaces for contamination and, if necessary, perform localized disinfection using integrated UV-light technology. This method enhances hygiene awareness and cleanliness in both personal and professional environments.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “smart device with integrated UV-light” and “device” are interchangeable and refer to the smart device with integrated UV-light 100 of the present invention.

Notwithstanding the foregoing, the smart device with integrated UV-light 100 of the present invention and its various components can be of any suitable size and configuration as is known in the art without affecting the overall concept of the invention, provided that they accomplish the above-stated objectives. One of ordinary skill in the art will appreciate that the size, configuration, and material of the smart device with integrated UV-light 100 as shown in the FIGS. are for illustrative purposes only, and that many other sizes and shapes of the smart device with integrated UV-light 100 are well within the scope of the present disclosure. Although the dimensions of the smart device with integrated UV-light 100 are important design parameters for user convenience, the smart device with integrated UV-light 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A smart device with integrated UV-light comprising:
a smart device comprising a processor, a memory unit, a rechargeable battery, and a communication module;
a UV-light module integrated into the smart device, the UV-light module configured to emit ultraviolet light; and
a software interface executable on the smart device, the software interface configured to control an activation of the UV-light module.

2. The smart device with integrated UV-light of claim 1, wherein the ultraviolet light is comprised of a UVA wavelength.

3. The smart device with integrated UV-light of claim 1, wherein the ultraviolet light is comprised of a UVB wavelength.

4. The smart device with integrated UV-light of claim 1, wherein the ultraviolet light is comprised of a UVC wavelength.

5. The smart device with integrated UV-light of claim 1, wherein the software interfaces adjusts a wavelength of the UV-light module.

6. The smart device with integrated UV-light of claim 1, wherein the UV-light module is integrated into a camera module of the smart device.

7. The smart device with integrated UV-light of claim 1 further comprised of an optical filter.

8. The smart device with integrated UV-light of claim 1, wherein the smart device is comprised of a button that activates the UV-light module.

9. A smart device with integrated UV-light comprising:
a smart device with integrated UV-light comprising:
a smart device comprising a processor, a memory unit, a rechargeable battery, and a communication module;
a UV-light module arranged in a ring configuration around a camera of the smart device, the UV-light module configured to emit ultraviolet light; and
a software interface executable on the smart device, the software interface configured to control an activation of the UV-light module.

10. The smart device with integrated UV-light of claim 9, wherein the ultraviolet light is comprised of a UVA wavelength between 315 and 400 nanometers.

11. The smart device with integrated UV-light of claim 9, wherein the ultraviolet light is comprised of a UVB wavelength between 280 and 315 nanometers.

12. The smart device with integrated UV-light of claim 9, wherein the ultraviolet light is comprised of a UVC wavelength 200 and 280 nanometers.

13. The smart device with integrated UV-light of claim 9 further comprised of an optical filter.

14. The smart device with integrated UV-light of claim 9, wherein an intensity of the UV-light module can be configured by the software interface.

15. The smart device with integrated UV-light of claim 9, wherein a wavelength of the UV-light module can be configured by the software interface.

16. The smart device with integrated UV-light of claim 9 further comprised of an optical filter.

17. The smart device with integrated UV-light of claim 9, wherein the smart device is comprised of a button that activates the UV-light module.

18. A method of using a smart device with integrated UV-light, the method comprising the following steps:
providing a smart device with integrated UV-light comprised of a smart device comprising a UV-light module, a camera module, a flashlight, and a software interface;
activating the UV-light module via the software interface or a physical button on the smart device;
selecting a UV wavelength of the UV-light module via the software interface; and
directing the smart device toward a target surface to illuminate the surface with an ultraviolet light from the UV-light module.

19. The method of using a smart device with integrated UV-light of claim 18, wherein the smart device is comprised of a smartphone or a tablet.

20. The method of using a smart device with integrated UV-light of claim 18, wherein the UV wavelength is comprised of a UVA, UVB, or a UVC.
